(19) United States
(12) Patent Application Publication
(10) Pub. No.: US 2025/0114023 A1
(43) Pub. Date: Apr. 10, 2025

(54) BIOSIGNAL MEASUREMENT ELECTRODE AND METHOD FOR PRODUCING SAME

(71) Applicant: Research & Business Foundation Sungkyunkwan University, Suwon-si (KR)

(72) Inventors: Tae il KIM, Suwon-si (KR); Joo Hwan SHIN, Suwon-si (KR); Ji Yeong CHOI, Suwon-si (KR)

(73) Assignee: Research & Business Foundation Sungkyunkwan University, Suwon-si (KR)

(21) Appl. No.: 18/903,489

(22) Filed: Oct. 1, 2024

(30) Foreign Application Priority Data
Oct. 4, 2023 (KR) 10-2023-0132078

(51) Int. Cl. A61B 5/268; A61B 5/257

(52) U.S. Cl. CPC A61B 5/268; A61B 5/257; A61B 2562/125

(57) ABSTRACT

Provided is a method for producing a biosignal measurement electrode including: preparing a conductive polymer including a coil-type chain structure having a coil shape; providing a sugar alcohol and a water-soluble polymer to the conductive polymer to produce an electrode source including an expanded chain structure in which the coil-type chain structure is expanded and linearly arranged; forming a coating layer by providing the electrode source on a substrate; and producing the biosignal measurement electrode by annealing the coating layer.

### BACKGROUND OF THE INVENTION

### 1. Field of the Invention

The present application relates to a biosignal measurement electrode and a method for producing the same, and more specifically, to a biosignal measurement electrode having excellent conductivity, flexibility, and adhesion, and a method for producing the same.

### 2. Description of the Related Art

Recently, as a wearable electronic device technology has developed, a biosignal measurement device that is attached to the skin to measure a biosignal generated in the body has been developed. The biosignal measurement device may measure an electrical signal generated in the body, for example, an electroencephalogram (EEG) signal, an electrocardiogram (ECG) signal, and an electromyogram (EMG) signal. In addition, the biosignal measurement device may measure a physiological signal generated in the body, for example, a signal using a metabolite such as uric acid, lactic acid, and glucose, and/or a signal using sweat. When the biosignal is monitored using the biosignal measurement device, time and/or space constraints may be reduced, whereas the amount of health care information that may be obtained may increase.

People can do various activities based on physical movements in their daily lives. Accordingly, when the biosignal measurement device is attached to the skin, a stimulus may be applied to the biosignal measurement device according to the movement of a body part to which the biosignal measurement device is attached. Meanwhile, the biosignal measurement device may include an electrode, and a stimulus may also be applied to an electrode of the biosignal measurement device, that is, a biosignal measurement electrode, according to the movement of the body. When the stimulus is applied in this way, the biosignal measurement electrode attached to the skin may be detached from the skin. Therefore, in order to measure a biosignal, the biosignal measurement electrode may require excellent adhesion with the skin even when the biosignal measurement electrode is attached to the skin to generate a stimulus in daily life.

Meanwhile, when flexibility of the biosignal measurement electrode is insufficient, noise may be generated in the biosignal measurement electrode according to the movement of the body part to which the biosignal measurement device is attached. Accordingly, signal measurement quality of the biosignal measurement device may be deteriorated. Therefore, excellent flexibility and/or conductivity may be required in the biosignal measurement electrode.

There is an increase in Research on a biosignal measurement electrode based on poly(3,4-ethylenedioxythiophene):polystyrenesulfonate (PEDOT:PSS), which is a material having adhesion, flexibility, and conductivity required for the above-described biosignal measurement electrode. For example, Korean Patent Registration No. 10-1732814 discloses a method for producing an electrode including: forming a dielectric layer on a gate and modifying a surface to be hydrophobic by applying a compound having a hydrophobic terminal group; selectively hydrophilizing the hydrophobically modified surface to obtain a surface having mutually different wettability; and dropping a PEDOT:PSS solution doped with HAuCl4 on the surface having mutually different wettability to form PEDOT:PSS only in a hydrophilized region, thereby obtaining a patterned PEDOT:PSS source/drain electrode, wherein the selective hydrophilization of the obtaining of the surface is performed by irradiating UV/ozone to the hydrophobically modified surface through a shadow mask, the PEDOT:PSS solution doped with HAuCl4 is prepared by adding HAuCl4 to a PEDOT:PSS solution to oxidize and dope PEDOT:PSS, and adding HAuCl4 such that a concentration of HAuCl4 in the entire solution becomes 10 mM, when the PEDOT:PSS solution doped with HAuCl4 is prepared, aggregation or precipitation of PEDOT:PSS does not occur, Au nanoparticles are not formed, an organic thin film transistor (OTFT) is a bottom-contact organic thin film transistor (OTFT) in which the PEDOT:PSS source/drain electrode is positioned under an organic semiconductor layer, and the organic semiconductor layer is a poly(3-hexylthiophene) (P3HT) thin film.

However, the PEDOT:PSS-based electrode may have limitations in use in real life. Specifically, when the sweat is secreted onto the skin, the adhesion of the PEDOT:PSS-based electrode may be greatly deteriorated. Accordingly, it may be difficult to continuously monitor the PEDOT:PSS-based electrode. That is, the PEDOT:PSS-based electrode may have a limitation in terms of the adhesion.

Meanwhile, the PEDOT:PSS-based electrode has excellent flexibility than metal, but may have an elongation at break of about 5%. The elongation at break of about 5% may be a very low value compared to about 50% of skin flexibility. A difference between the flexibility of the PEDOT:PSS-based electrode and the flexibility of the skin may cause stress to the electrode and/or the skin, or may make continuous monitoring difficult. That is, the PEDOT:PSS-based electrode may have a limitation in terms of the flexibility.

Meanwhile, the PEDOT:PSS-based electrode may have lower conductivity in a pure state where the amount of additives included decreases. That is, the pure PEDOT:PSS-based electrode that does not include the additives may have a limitation in terms of the conductivity. Thus, the PEDOT:PSS-based electrode may have a limitation in measuring a biosignal.

Accordingly, there is a demand for a biosignal measurement electrode having excellent properties in terms of adhesion, flexibility, and conductivity.

### SUMMARY OF THE INVENTION

One technical problem to be solved by the present application is to provide a biosignal measurement electrode having excellent adhesion, and a method for producing the same.

Another technical problem to be solved by the present application is to provide a biosignal measurement electrode having excellent flexibility, and a method for producing the same.

Still another technical problem to be solved by the present application is to provide a biosignal measurement electrode having excellent charge transport properties, and a method for producing the same.

Still another technical problem to be solved by the present application is to provide a biosignal measurement electrode in which a form attached to the skin is maintained even in external stimuli such as exercise and daily life, and has excellent biosignal measurement ability, and a method for producing the same.

The technical problems to be solved by the present application are not limited to those described above.

In order to solve the technical problems, the present application provides a method for producing a biosignal measurement electrode.

According to one embodiment, the method for producing a biosignal measurement electrode may include: preparing a conductive polymer including a coil-type chain structure having a predominant coil shape, that is, a benzoid PEDOT resonance structure; producing an electrode source including a conductive polymer in which the coil-type chain structure, that is, the benzoid PEDOT structure is expanded and linearly arranged, that is, the coil-type chain structure, that is, the benzoid PEDOT structure is converted into a quinoid PEDOT structure, by providing the conductive polymer with a sugar alcohol and a water-soluble polymer; forming a coating layer by providing the electrode source on a substrate; and producing the biosignal measurement electrode by annealing the coating layer.

According to one embodiment, the sugar alcohol may have a hydroxyl group, and may form a hydrogen bond with a sulfonate group of PSS, which is a non-conductive region of the conductive polymer, to convert the coil-type chain structure of conductive PEDOT into a coil-type or linear chain structure.

According to one embodiment, the conductive polymer of the expanded coil-type chain structure may have an amorphous structure, and the water-soluble polymer may have a linear structure and may be bonded to the conductive polymer having the expanded coil-type chain structure, that is, having the amorphous structure, so as to linearly rearrange the disordered coil-type chain structure according to the linear structure.

According to one embodiment, when the biosignal measurement electrode is attached to the skin, the water-soluble polymer may be gelled have a swelling force by reacting with a body fluid, and the sugar alcohol may form a hydrogen bond with the conductive polymer and the water-soluble polymer to increase the swelling force of the water-soluble polymer and decrease a degree of gelation.

According to one embodiment, in the preparing of the conductive polymer, the conductive polymer may have a benzoid structure, and in the producing of the electrode source, the benzoid structure may be converted into a quinoid structure.

According to one embodiment, the producing of the electrode source may include: producing a mixed solution including 10 wt % or greater to 20 wt % or less of a mixture of the sugar alcohol and the water-soluble polymer; and providing 10 wt % or greater to 30 wt % or less of the mixed solution to a solution in which the conductive polymer is dispersed in an amount of 0.1 at wt % or greater to 3 wt % or less, and stirring the mixed solution.

According to one embodiment, the sugar alcohol may be provided in an amount of 50 wt % or greater to 70 wt % or less based on 100 wt % of the mixture.

In order to solve the technical problems, the present application provides a biosignal measurement electrode.

According to one embodiment, the biosignal measurement electrode may include: a conductive polymer; a sugar alcohol; and a water-soluble polymer, in which the biosignal measurement electrode may include an expanded chain structure in which the conductive polymer, the sugar alcohol, and the water-soluble polymer are bonded, and the expanded chain structure may be more expanded than a coil-type chain structure having a coil shape of the conductive polymer before the bonding, and may be linearly arranged.

According to one embodiment, the expanded chain structure may have more non-localized electrons along a linear conjugated pi electromagnetic system than the coil-type chain structure, so that charge transport properties may be improved.

According to one embodiment, when the biosignal measurement electrode is attached to the skin, the water-soluble polymer may form a hydrogen bond with a body fluid to provide adhesion, and the sugar alcohol may form a hydrogen bond with the conductive polymer and the water-soluble polymer to provide flexibility.

According to one embodiment, in a range of 1439 cm⁻¹ or greater to 1423 cm⁻¹ or less, which is defined as a C_{α}=C_{β} antisymmetric vibration peak, the biosignal measurement electrode may have a Raman spectrum peak which shifts to a wavenumber lower than a wavenumber of the conductive polymer.

According to one embodiment, the biosignal measurement electrode may include the water-soluble polymer and the sugar alcohol in a weight ratio of 4:6.

According to one embodiment, the conductive polymer may include poly3,4-ethylenedioxythiophene:polystyrene sulfonate, the water-soluble polymer may include at least one of polyvinyl alcohol, polyethylene oxide, polyacryl amide, polyvinyl pyrrolidone, polyacrylic acid, polystyrenesulfonic acid, polysilicic acid, polyphosphoric acid, polyethylenesulfonic acid, polymaleic acid, polyamines, polyacrylamide, poly polyvinylpyrrolidone, and polyethylene glycol, and the sugar alcohol may include at least one of sorbitol, ethylene glycol, glycerol, erythritol, threitol, arabitol, xylitol, ribitol, mannitol, galactitol, fucitol, iditol, inositol, volemitol, isomalt, maltitol, lactitol, maltotritol, maltotetraitol, and polyglycitol.

In order to solve the technical problems, the present application provides a biosignal measurement electrode.

According to one embodiment, the biosignal measurement electrode may include a first layer attached to a skin; a second layer stacked on the first layer; and a third layer stacked on the second layer, in which the first to third layers may include an expanded chain structure in which a conductive polymer, a sugar alcohol, and a water-soluble polymer are bonded, and the expanded chain structure may be more expanded than a coil-type chain structure having a coil structure of the conductive polymer before the bonding, and may be linearly arranged.

According to one embodiment, the first layer may include a larger amount of the water-soluble polymer in comparison with the second layer and the third layer, and may include a smaller amount of the sugar alcohol in comparison with the second layer and the third layer, the second layer may include a smaller amount of the water-soluble polymer in comparison with the first layer and a larger amount of the water-soluble polymer in comparison with the third layer, and may include a larger amount of the sugar alcohol in comparison with the first layer and a smaller amount of the sugar alcohol in comparison with the third layer, and the third layer may include a smaller amount of the water-soluble polymer in comparison with the first layer and the second layer, and may include a larger amount of the sugar alcohol in comparison with the first layer and the second layer.

The method for producing a biosignal measurement electrode according to the embodiment of the present application may include: preparing a conductive polymer including a coil-type chain structure having a coil shape; producing an electrode source including an expanded chain structure in which the coil-type chain structure is expanded and linearly arranged by providing the conductive polymer with a sugar alcohol and a water-soluble polymer; forming a coating layer by providing the electrode source on a substrate; and producing the biosignal measurement electrode by annealing the coating layer.

The expanded chain structure may have a larger amount of non-localized electrons along a linear conjugated pi electromagnetic system in comparison with the coil-type chain structure. Accordingly, the biosignal measurement electrode may have excellent charge transport properties.

When the biosignal measurement electrode is attached to the skin, the water-soluble polymer may be gelled and may have a swelling force by reacting with a body fluid. Accordingly, the biosignal measurement electrode may have excellent adhesion.

The sugar alcohol may form a hydrogen bond with the conductive polymer and the water-soluble polymer. Accordingly, the biosignal measurement electrode may have excellent flexibility.

According to a specific embodiment of the present application, the biosignal measurement electrode may include the water-soluble polymer and the sugar alcohol in a weight ratio of 4:6. Accordingly, the biosignal measurement electrode may have excellent adhesion and flexibility. This may be because, in the embodiment of the present application, the sugar alcohol provides flexibility to the biosignal measurement electrode, and the water-soluble polymer provides adhesion to the biosignal measurement electrode. More specifically, when the biosignal measurement electrode is attached to the skin, a hydrogen bond is made with the lipid and protein of the epidermis of the skin during gelation and drying of the water-soluble polymer by the body fluid over time, so that adhesion between the biosignal measurement electrode and the skin may be increased. Meanwhile, the adhesion between the biosignal measurement electrode and the skin may be maintained for a long time by the sugar alcohol having excellent hygroscopicity. Accordingly, the biosignal measurement electrode may have excellent long-time use stability. Meanwhile, when the skin is stretched or compressed in a state where the biosignal measurement electrode is attached to the skin, the biosignal measurement electrode having excellent flexibility may have substantially the same flexibility as the skin, and may thus move according to the movement of the skin and maintain close contact with the skin. Accordingly, the biosignal measurement electrode may effectively measure a bio-electrical signal and a bio-physiological signal in the long term.

According to the embodiment of the present application, since the biosignal measurement electrode has excellent adhesion, a substantial contact area with the curved skin may be maximized. Therefore, unlike a conventional electrode that is produced to be thin or produced in the form of serpentine in order to secure flexibility, the biosignal measurement electrode has an advantage that the entire maximum area thereof may be used as a skin attachment area as it is. Therefore, according to the present application, since the biosignal measurement electrode is attached to the skin to minimize impedance, signal quality thereof may be excellent.

In addition, since the biosignal measurement electrode has excellent adhesion and flexibility as described above, the biosignal measurement electrode may be attached to the user's skin to easily maintain electrical and mechanical properties even when the skin is stretched according to the user's movement. Accordingly, the biosignal measurement electrode may have excellent signal quality.

In addition, since the biosignal measurement electrode has excellent adhesion and flexibility as described above, when the biosignal measurement electrode is attached to the skin, a gap between the biosignal measurement electrode and the skin may be minimized, and accordingly, noise due to an external force, for example, external vibration, may be minimized.

In addition, since the biosignal measurement electrode provides excellent adhesion by reacting with the body fluid as described above, long-term use stability may be excellent.

### BRIEF DESCRIPTION OF THE DRAWINGS

FIG. 1 is a view for explaining a method for producing a biosignal measurement electrode according to an embodiment of the present application.

FIG. 2 is a view for explaining a step of preparing a conductive polymer in the method for producing a biosignal measurement electrode according to the embodiment of the present application.

FIG. 3 is a view for explaining a step of producing an electrode in the method for producing a biosignal measurement electrode according to the embodiment of the present application.

FIG. 4 is a view for explaining a step of forming a coating layer and a step of producing a biosignal measurement electrode in the method for producing a biosignal measurement electrode according to the embodiment of the present application.

FIG. 5 is a view for explaining a biosignal measurement electrode according to a modification example of the present application.

FIG. 6 is a Fourier transform infrared spectroscopy measurement graph of Experimental Example 1, a water-soluble polymer, Experimental Example 2, and Experimental Example 7 of the present application.

FIG. 7 is a Raman spectroscopy measurement graph of Experimental Examples 1 to 10 of the present application.

FIG. 8 is a conductivity measurement graph of Experimental Examples 2 to 10 of the present application.

FIGS. 9A to 9D are surface profile data when Experimental Example 2, Experimental Example 3, Experimental Example 6, and Experimental Example 9 of the present application are stretched by 50%.

FIG. 10 is a graph of measuring a wavelength and an amplitude of wrinkles formed when Experimental Examples 2 to 9 of the present application are stretched by 50%.

FIG. 11 is a graph of measuring a tensile strength of Experimental Examples 3 to 9 of the present application.

FIG. 12 is a graph showing test results of adhesion measured by attaching Experimental Examples 2 to 10 of the present application to the skin.

FIG. 13 is a graph showing impedance measured by attaching a commercially available electrode, Experimental Example 7 of the present application, a gold-based electrode, and Experimental Example 1 to the skin.

FIG. 14 is a graph showing impedance measured by attaching the commercially available electrode, Experimental Example 7 of the present application, the gold-based electrode, and Experimental Example 1 to the skin and applying various stimuli.

FIGS. 15A to 15C are graph showing changes in impedance measured by attaching the commercially available electrode, Experimental Example 7 of the present application, the gold-based electrode, and Experimental Example 1 to the skin and exercising.

FIG. 16 is a photograph taken after attaching the commercially available electrode, Experimental Example 7 of the present application, the gold-based electrode, and Experimental Example 1 to the skin and exercising.

FIG. 17 is a photograph taken for 7 days while attaching Experimental Example 7 of the present application to the skin and living a daily life.

FIG. 18 is a graph of changes in impedance measured for 7 days by attaching Experimental Example 7 of the present application and the commercially available electrode to the skin and living a daily life.

FIG. 19 is a graph of a biosignal measured by attaching Experimental Example 7 of the present application to the skin.

### DETAILED DESCRIPTION OF THE INVENTION

Hereinafter, preferred embodiments of the present invention will be described in detail with reference to the accompanying drawings. However, the present invention may be embodied in different forms and should not be construed as limited to the embodiments set forth herein. Rather, the embodiments introduced herein are provided so that the disclosed contents may be thorough and complete and the spirit of the present invention may be sufficiently conveyed to those skilled in the art.

In the present specification, it will be understood that when an element is referred to as being “on” another element, it can be formed directly on the other element or intervening elements may be present. In the drawings, the thicknesses of layers and regions are exaggerated for clarity.

In addition, it will be also understood that although the terms first, second, third, etc. may be used herein to describe various elements, these elements should not be limited by these terms. These terms are only used to distinguish one element from another element. Thus, a first element in some embodiments may be termed a second element in other embodiments without departing from the teachings of the present invention. Embodiments explained and illustrated herein include their complementary counterparts. As used herein, the term “and/or” includes any and all combinations of one or more of the associated listed elements.

The singular expression also includes the plural meaning as long as it does not differently mean in the context. In addition, the terms “comprise”, “have” etc., of the description are used to indicate that there are features, numbers, steps, elements, or combination thereof, and they should not exclude the possibilities of combination or addition of one or more features, numbers, operations, elements, or a combination thereof. Furthermore, it will be understood that when an element is referred to as being “connected” or “coupled” to another element, it may be directly connected or coupled to the other element or intervening elements may be present.

In addition, when detailed descriptions of related known functions or constitutions are considered to unnecessarily cloud the gist of the present invention in describing the present invention below, the detailed descriptions will not be included.

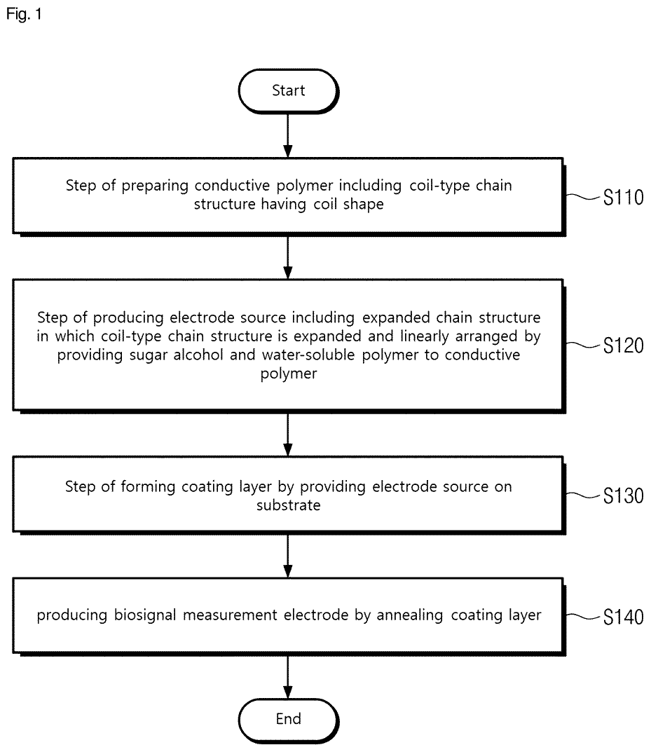

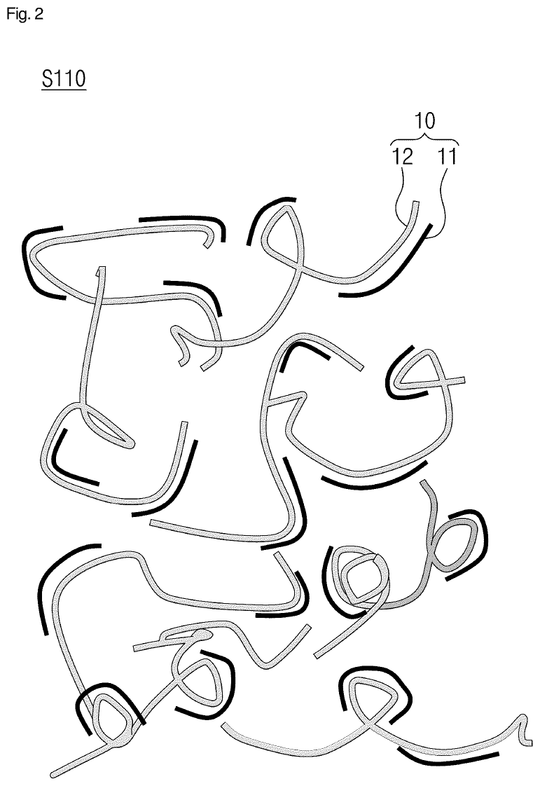

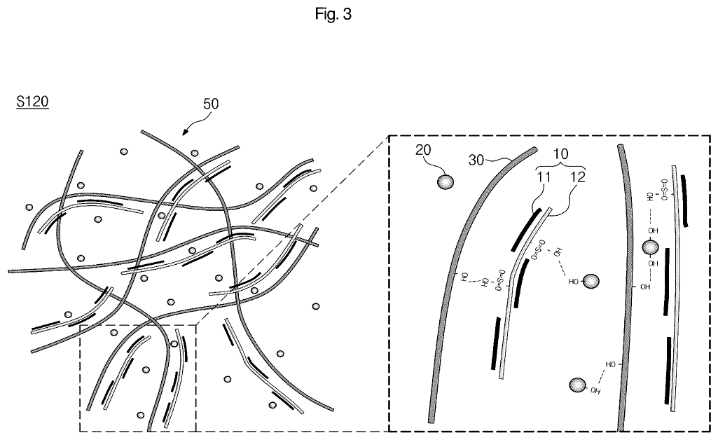

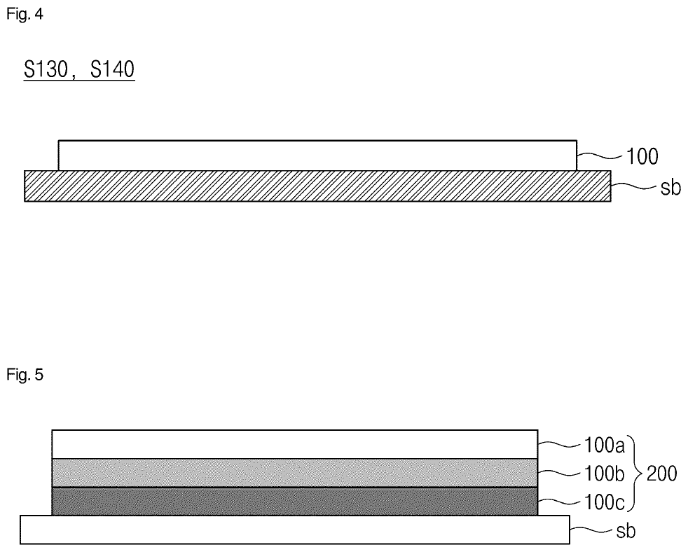

FIG. 1 is a view for explaining a method for producing a biosignal measurement electrode according to an embodiment of the present application, FIG. 2 is a view for explaining a step of preparing a conductive polymer in the method for producing a biosignal measurement electrode according to the embodiment of the present application, FIG. 3 is a view for explaining a step of producing an electrode in the method for producing a biosignal measurement electrode according to the embodiment of the present application, and FIG. 4 is a view for explaining a step of forming a coating layer and a step of producing a biosignal measurement electrode in the method for producing a biosignal measurement electrode according to the embodiment of the present application.

Referring to FIGS. 1 and 2, a conductive polymer 10 including a coil-type chain structure having a coil shape may be prepared (S110).

According to one embodiment, the conductive polymer 10 may include non-localized electrons along a linear conjugated pi electron system. Accordingly, the conductive polymer 10 may have excellent charge transport properties. Meanwhile, the conductive polymer 10 may have a benzoid structure. That is, this may mean that the conductive polymer 10 includes the coil-type chain structure. Meanwhile, when the coil-type chain structure is expanded, the benzoid structure may be converted into a quinoid structure. As described above, when the benzoid structure is converted into the quinoid structure, the non-localized electrons may exist along the linear conjugated pi electron system, and thus charge transport properties may be improved.

According to one embodiment, the conductive polymer 10 may include a first polymer 11 and a second polymer 12, which are different from each other. The first polymer 11 may have conductivity, and the second polymer 12 may have non-conductivity. That is, the conductive polymer 10 may have conductivity by the first polymer 11. For example, the conductive polymer 10 may include poly3,4-ethylenedioxythiophene (PEDOT) as the first polymer 11 and polystyrene sulfonate (PSS) as the second polymer 12. That is, the conductive polymer 10 may include, for example, poly3, 4-ethylenedioxythiophene:polystyrene sulfonate (PEDOT:PSS). As shown in FIG. 2, the conductive polymer 10 may include a coil-type chain structure.

Referring to FIGS. 1 and 3, the conductive polymer 10 may be provided with a sugar alcohol 20 and a water-soluble polymer 30 so as to produce an electrode source 50 including an expanded chain structure in which the coil-type chain structure is expanded and linearly arranged (S120).

According to one embodiment, the sugar alcohol 20 may have a hydroxyl group (OH), and a hydrogen bond with the non-conductive region of the conductive polymer 1 may be formed through the hydroxyl group (OH). That is, the sugar alcohol 20 may form a hydrogen bond with the second polymer 12 of the conductive polymer 10, that is, a sulfonate group of PSS, through the hydroxyl group (OH). Accordingly, the coil-type chain structure of the conductive polymer 10 may be expanded. In this case, the conductive polymer 10 of the expanded coil-type chain structure may have an amorphous structure.

According to one embodiment, the sugar alcohol 20 may include at least one of sorbitol, ethylene glycol, glycerol, erythritol, threitol, arabitol, xylitol, ribitol, mannitol, galactitol, fucitol, iditol, inositol, volemitol, isomalt, maltitol, lactitol, maltotritol, maltotetraitol, and polyglycitol.

According to one embodiment, the water-soluble polymer 30 may have a linear structure and may be bonded with the conductive polymer 10 having the expanded coil-type chain structure so as to linearly rearrange the coil-type chain structure according to the linear structure. In other words, the water-soluble polymer 30 may be bonded with the conductive polymer 10 having the amorphous structure so as to linearly rearrange a disordered structure of the conductive polymer 10 according to the linear structure.

According to one embodiment, when the biosignal measurement electrode is attached to a skin, the water-soluble polymer 30 may be gelled and have a swelling force by reacting with the body fluid. Accordingly, when the biosignal measurement electrode 100 produced including the water-soluble polymer 30 is adhered to the skin, adhesion may be excellent. Meanwhile, the sugar alcohol 20 may form a hydrogen bond with the conductive polymer 10 and the water-soluble polymer 30, thereby increasing the swelling force of the water-soluble polymer 30 and decreasing a degree of gelation. Accordingly, when the biosignal measurement electrode 100 is adhered to the skin, long-time use stability may be improved. This may be because, when the biosignal measurement electrode 100 is attached to the skin, the water-soluble polymer 30 reacts with the body fluid to provide adhesion, and the sugar alcohol 20 forms a hydrogen bond with the conductive polymer 10 and the water-soluble polymer 30 to provide flexibility.

According to one embodiment, the water-soluble polymer 30 may include at least one of polyvinyl alcohol, polyethylene oxide, polyacryl amide, polyvinyl pyrrolidone, polyacrylic acid, polystyrenesulfonic acid, polysilicic acid, polyphosphoric acid, polyethylenesulfonic acid, polymaleic acid, polyamines, polyacrylamide, poly. polyethylene glycol, and

According to one embodiment, the step of producing the electrode source 50 may include a step of producing a mixed solution including 10 wt % or greater to 20 wt % or less of a mixture of the sugar alcohol and the water-soluble polymer. For example, the mixed solution may be produced to include 15 wt % of the mixture of the sugar alcohol 20 and the water-soluble polymer 30. In this case, the sugar alcohol 20 may be provided in an amount of 50 wt % or greater to 70 wt % or less based on 100 wt % of the mixture. For example, the mixture may include 50 wt % of the water-soluble polymer 30 and 50 wt % of the sugar alcohol 20. For another example, the mixture may include 40 wt % of the water-soluble polymer 30 and 60 wt % of the sugar alcohol 20. For another example, the mixture may include 30 wt % of the water-soluble polymer 30 and 70 wt % of the sugar alcohol 20. When a weight ratio of the sugar alcohol 20 in the mixture increases, the flexibility of the biosignal measurement electrode 100 to be produced may be improved. Meanwhile, when a weight ratio of the water-soluble polymer 30 in the mixture increases, the adhesion of the biosignal measurement electrode 100 to be produced may be improved. That is, according to the embodiment of the present application, the conductivity may be provided by the conductive polymer 10 and the weight ratio of the sugar alcohol 20 and the water-soluble polymer 30 included in the mixture may be adjusted, thereby controlling the flexibility and the adhesion of the biosignal measurement electrode 100 to be produced. That is, according to the embodiment of the present application, since the weight ratio of the sugar alcohol 20 and the water-soluble polymer 30 may be adjusted, there is an advantage in that the biosignal measurement electrode 100 may be produced by appropriately controlling the flexibility and the adhesion for each region of the skin.

Further, the step of producing the electrode source 50 may include a step of providing 10 wt % or greater to 30 wt % or less of the mixed solution to a solution in which the conductive polymer 10 is dispersed in an amount of 0.1 wt % or greater to 3 wt % or less, and stirring the mixed solution. For example, the electrode source 50 may be produced by providing 20 wt % of the mixed solution to a solution in which of the conductive polymer 10 is dispersed in an amount of 1.3 wt %, and stirring the mixed solution at room temperature for 3 hours. Accordingly, the benzoid structure may be converted into the quinoid structure. That is, this may mean that the electrode source 50 includes the expanded chain structure in which the coil-type chain structure is expanded and linearly arranged. Meanwhile, as described above, the conductive polymer 10 may have the non-localized electrons along the linear conjugated pi electron system, and thus charge transport properties thereof may be improved. When the coil-type chain structure is expanded so that the benzoid structure is converted into the quinoid structure, the quinoid structure may have a larger amount of non-localized electrons along the linear conjugated pi electron system in comparison with the benzoid structure. Accordingly, the biosignal measurement electrode 100 to be produced may have excellent charge transport properties.

Referring to FIGS. 1 and 4, the electrode source 50 may be provided on a substrate sb to form a coating layer (not shown) (S130).

The coating layer may be produced by providing the electrode source 50 on the substrate sb using any one method selected from, for example, spin coating, dip-coating, doctor blade, metering rod, slot-casting, spray coating, screen printing, inkjet printing, and aerogel jet.

Continuing to refer to FIGS. 1 and 4, the coating layer (not shown) may be annealed to produce the biosignal measurement electrode 100 (S140).

According to one embodiment, the annealing may be performed at 90° C. or higher and 150° C. or lower for 10 minutes or longer and 60 minutes or shorter. For example, the annealing may be performed at 110° C. for 20 minutes. Accordingly, the biosignal measurement electrode 100 to be produced may have excellent conductivity and flexibility. On the other hand, when the annealing is performed at higher than 150° C. for longer than 60 minutes, the conductivity of the biosignal measurement electrode to be produced may be improved, but the flexibility may be reduced. Alternatively, when the annealing is performed at lower than 90° C. for shorter than 10 minutes, the flexibility of the biosignal measurement electrode to be produced may be improved, but the conductivity may be deteriorated. However, according to the embodiment of the present application, the annealing may be performed at 90° C. or higher and 150° C. or lower for 10 minutes or longer and 60 minutes or shorter, and thus the conductivity and flexibility of the biosignal measurement electrode 100 to be produced may be improved.

Referring to FIG. 4, the biosignal measurement electrode 100 may include at least one of the conductive polymer 10, the sugar alcohol 20, and the water-soluble polymer 30.

According to one embodiment, the biosignal measurement electrode 100 may include an expanded chain structure in which the conductive polymer 10 sugar alcohol 20, and the water-soluble polymer 30 are bonded. Meanwhile, the expanded chain structure may be more expanded than the coil-type chain structure having a coil shape of the conductive polymer 10 before the bonding, and may be linearly arranged. The expanded chain structure may have a larger amount of non-localized electrons along a linear conjugated pi electromagnetic system in comparison with the coil-type chain structure. Accordingly, the biosignal measurement electrode 100 may have excellent charge transport properties. In this regard, the description of the above-described embodiment will be referred to.

According to one embodiment, the biosignal measurement electrode 100 may include the water-soluble polymer 30 and the sugar alcohol 20 in a weight ratio of 4:6. Accordingly, the biosignal measurement electrode 100 may have excellent adhesion and flexibility. This may be because, in the embodiment of the present application, the sugar alcohol 20 provides flexibility to the biosignal measurement electrode 100, and the water-soluble polymer 30 provides adhesion to the biosignal measurement electrode 100. More specifically, when the biosignal measurement electrode 100 is attached to the skin, the water-soluble polymer 30 may be gelled by the body fluid over time, so that the adhesion between the biosignal measurement electrode 100 and the skin may be enhanced. Meanwhile, the adhesion between the biosignal measurement electrode 100 and the skin may be maintained for a long time by the sugar alcohol 20 having excellent hygroscopicity. Accordingly, the biosignal measurement electrode 100 may have excellent long-time use stability. Meanwhile, when the skin is stretched or compressed in a state where the biosignal measurement electrode 100 is attached to the skin, the biosignal measurement electrode 100 having excellent flexibility may have substantially the same flexibility as the skin, and may thus move according to the movement of the skin and maintain close contact with the skin. Accordingly, the biosignal measurement electrode 100 may effectively measure a bio-electrical signal and a bio-physiological signal in the long term.

According to the embodiment of the present application, since the biosignal measurement electrode 100 has excellent adhesion, a substantial contact area with the curved skin may be maximized. Therefore, unlike a conventional electrode that is produced to be thin or produced in the form of serpentine in order to secure flexibility, the biosignal measurement electrode 100 has an advantage that the entire maximum area thereof may be used as a skin attachment area as it is. Therefore, according to the present application, since the biosignal measurement electrode 100 is attached to the skin to minimize impedance, signal quality thereof may be excellent.

In addition, since the biosignal measurement electrode 100 has excellent adhesion and flexibility as described above, the biosignal measurement electrode 100 may be attached to the user's skin to easily maintain electrical and mechanical properties even when the skin is stretched according to the user's movement. Accordingly, the biosignal measurement electrode 100 may have excellent signal quality.

In addition, since the biosignal measurement electrode 100 has excellent adhesion and flexibility as described above, when the biosignal measurement electrode is attached to the skin, a gap between the biosignal measurement electrode and the skin may be minimized, and accordingly, noise due to an external force, for example, external vibration, may be minimized.

In addition, since the biosignal measurement electrode 100 provides excellent adhesion by reacting with the body fluid as described above, long-term use stability may be excellent.

Hereinafter, a modification example of the present application will be described.

FIG. 5 is a view for explaining a biosignal measurement electrode according to a modification example of the present application.

Referring to FIG. 5, a biosignal measurement electrode 200 according to the modification example of the present application may include: a first layer 100a attached to the skin; a second layer 100b stacked on the first layer 100a; and a third layer 100c stacked on the second layer 100b.

The first to third layers 100a to 100c may include an expanded chain structure in which the conductive polymer 10, the sugar alcohol 20, and the water-soluble polymer 30, which are described above, are bonded. Meanwhile, the expanded chain structure may be more expanded than the coil-type chain structure having a coil shape of the conductive polymer 10 before the bonding, and may be linearly arranged. In this regard, the description of the above-described embodiment will be referred to.

According to the modification example of the present application, the first layer 100a may include a larger amount of the water-soluble polymer 30 in comparison with the second layer 100b and the third layer 100c, and may include a smaller amount of the sugar alcohol 20 in comparison with the second layer 100b and the third layer 100c. The second layer 100b may include a smaller amount of the water-soluble polymer 30 in comparison with the first layer 100a and a larger amount of the water-soluble polymer 30 in comparison with the third layer 100c, and may include a larger amount of the sugar alcohol 20 in comparison with the first layer 100a and a smaller amount of the sugar alcohol 20 in comparison with the third layer 100c. The third layer 100c may include a smaller amount of the water-soluble polymer 30 in comparison with the first layer 100a and the second layer 100b, and may include a larger amount of the sugar alcohol 20 in comparison with the first layer 100a and the second layer 100b. For example, the first layer 100a may include the water-soluble polymer 30 and the sugar alcohol 20 in a weight ratio of 5:5. The second layer 100b may include the water-soluble polymer 30 and the sugar alcohol 20 in a weight ratio of 4:6. The third layer 100c may include the water-soluble polymer 30 and the sugar alcohol 20 in a weight ratio of 3:7.

As described above, since the first layer 100a includes the water-soluble polymer 30 having a higher weight ratio than the second and third layers 100b and 100c, the biosignal measurement electrode 200 may have excellent adhesion due to the first layer 100a. Meanwhile, since the second layer 100b includes the water-soluble polymer 30 and the sugar alcohol 20 in a weight ratio of 4:6, the biosignal measurement electrode 200 may have excellent conductivity due to the second layer 100b. Furthermore, since the second layer 100b mechanically has an intermediate modulus between the first layer 100a and the third layer 100c, an extreme modulus mismatch between the first and third layers 100a and 100c may be minimized. Therefore, the biosignal measurement electrode 200 may have excellent mechanical stability due to the second layer 100b. Meanwhile, since the third layer 100c includes the sugar alcohol 20 having a higher weight ratio than the first and second layers 100a and 100b, the biosignal measurement electrode 200 may have excellent flexibility due to the third layer 100c.

As a result, according to the modification example of the present application, since the biosignal measurement electrode 200 has adhesion through the first layer 100a attached to the skin, electrical conductivity of the second layer 100b may be stably maintained even when the biosignal measurement electrode 200 is stretched or contracted. In addition, since the biosignal measurement electrode 200 has flexibility through the third layer 100c provided on an outside, cracks may be minimized even when the biosignal measurement electrode 200 is stretched or contracted, thereby maintaining electrical conductivity of the second layer 100b. That is, the biosignal measurement electrode 200 according to the modification example of the present application may have excellent adhesion, conductivity, and flexibility.

Meanwhile, the biosignal measurement electrode 200 may be produced by the steps of providing the electrode source 50 including the water-soluble polymer 30 and the sugar alcohol 20 in a weight ratio of 3:7 on the substrate sb to form the third layer 100c, providing the electrode source 50 including the water-soluble polymer 30 and the sugar alcohol 20 in a weight ratio of 4:6 on the third layer 100c to form the second layer 100b, and providing the electrode source 50 including the water-soluble polymer 30 and the sugar alcohol 20 in a weight ratio of 5:5 on the second layer 100b to form the first layer 100a.

Hereinafter, specific experimental examples and characteristic evaluation results of the biosignal measurement electrode 100 according to the embodiment of the present application will be described.

Production of Biosignal Measurement Electrode (ex1) According to Experimental Example 1

PEDOT:PSS was prepared as the conductive polymer 10.

On a polydimethylsiloxane (PDMS) substrate sb, water in which the conductive polymer 10 was dispersed in an amount of 1.3 wt % was provided, and annealing was performed at 110° C. for 20 minutes to produce a biosignal measurement electrode (ex1) having a thickness of 10 μm according to Experimental Example 1.

Production of Biosignal Measurement Electrode (ex2) According to Experimental Example 2

In Experimental Example 1 described above, PEDOT:PSS was prepared as the conductive polymer 10, d-sorbitol, which is a hexavalent sugar alcohol, was prepared as the sugar alcohol 20, and PVA was prepared as the water-soluble polymer 30. In the present application, the materials used, that is, PEDOT:PS, d-sorbitol, and PVA, may be materials having biocompatibility.

The sugar alcohol 20 and the water-soluble polymer 30 were mixed with water so that a mixing amount of the mixture was 15 wt %, thereby producing the mixed solution. In this case, the weight ratio of the water-soluble polymer 30 and the sugar alcohol 20 in the mixture was adjusted to 10:0.

20 wt % of the mixed solution was provided to water in which the conductive polymer 10 was dispersed in an amount of 1.3 wt %, and stirred at room temperature for 3 hours to produce the electrode source 50.

The electrode source 50 was provided on the substrate sb, and annealing was performed at 110° C. for 20 minutes to produce a biosignal measurement electrode (ex2) according to Experimental Example 2.

Production of Biosignal Measurement Electrode (ex3) According to Experimental Example 3

The weight ratio of the water-soluble polymer 30 and the sugar alcohol 20 was adjusted to 8:2 in Experimental Example 2 described above to produce a biosignal measurement electrode (ex3) according to Experimental Example 3.

Production of Biosignal Measurement Electrode (ex4) According to Experimental Example 4

The weight ratio of the water-soluble polymer 30 and the sugar alcohol 20 was adjusted to 7:3 in Experimental Example 2 described above to produce a biosignal measurement electrode (ex4) according to Experimental Example 4.

Production of Biosignal Measurement Electrode (ex5) According to Experimental Example 5

The weight ratio of the water-soluble polymer 30 and the sugar alcohol 20 was adjusted to 6:4 in Experimental Example 2 described above to produce a biosignal measurement electrode (ex5) according to Experimental Example 5.

Production of Biosignal Measurement Electrode (ex6) According to Experimental Example 6

The weight ratio of the water-soluble polymer 30 and the sugar alcohol 20 was adjusted to 5:5 in Experimental Example 2 described above to produce a biosignal measurement electrode (ex6) according to Experimental Example 6.

Production of Biosignal Measurement Electrode (ex7) According to Experimental Example 7

The weight ratio of the water-soluble polymer 30 and the sugar alcohol 20 was adjusted to 4:6 in Experimental Example 2 described above to produce a biosignal measurement electrode (ex7) according to Experimental Example 7.

Production of Biosignal Measurement Electrode (ex8) According to Experimental Example 8

The weight ratio of the water-soluble polymer 30 and the sugar alcohol 20 was adjusted to 3:7 in Experimental Example 2 described above to produce a biosignal measurement electrode (ex8) according to Experimental Example 8.

Production of Biosignal Measurement Electrode (ex9) According to Experimental Example 9

The weight ratio of the water-soluble polymer 30 and the sugar alcohol 20 was adjusted to 2:8 in Experimental Example 2 described above to produce a biosignal measurement electrode (ex9) according to Experimental Example 9.

Production of Biosignal Measurement Electrode (ex10) According to Experimental Example 10

The weight ratio of the water-soluble polymer 30 and the sugar alcohol 20 was adjusted to 0:10 in Experimental Example 2 described above to produce a biosignal measurement electrode (ex10) according to Experimental Example 10.

Experimental Examples 1 to 10 described above may be summarized as shown in Table 1 below.

TABLE 1

Weight ratio

| | Water-soluble | Sugar |
|---|---|---|
| Classification | polymer 30 | alcohol 20 |
| Experimental Example 1 (ex1) | 0 | 0 |
| Experimental Example 2 (ex2) | 10 | 0 |
| Experimental Example 3 (ex3) | 8 | 2 |
| Experimental Example 4 (ex4) | 7 | 3 |
| Experimental Example 5 (ex5) | 6 | 4 |
| Experimental Example 6 (ex6) | 5 | 5 |
| Experimental Example 7 (ex7) | 4 | 6 |
| Experimental Example 8 (ex8) | 3 | 7 |
| Experimental Example 9 (ex9) | 2 | 8 |
| Experimental Example 10 (ex10) | 0 | 10 |

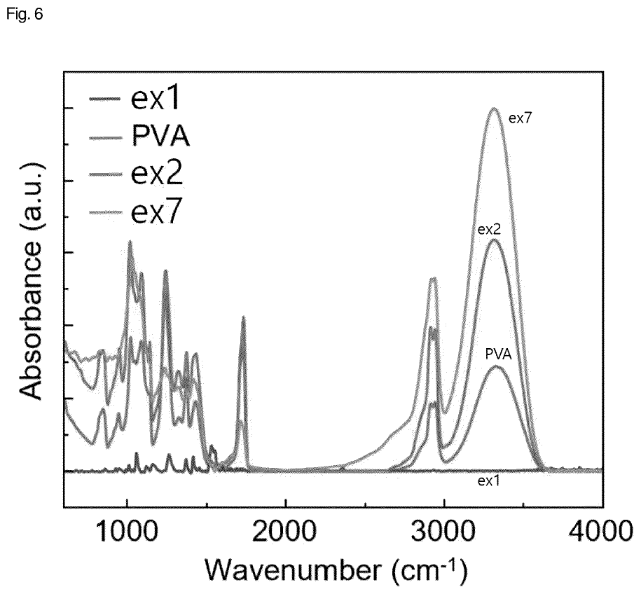

FIG. 6 is a Fourier transform infrared spectroscopy measurement graph of Experimental Example 1, a water-soluble polymer, Experimental Example 2, and Experimental Example 7 of the present application.

In FIG. 6, Experimental Example 1 (ex1) may mean a pure conductive polymer 10 that is not bonded to the water-soluble polymer 30 and the sugar alcohol 20 as in the above-described Experimental Examples. In addition, in FIG. 6, the water-soluble polymer may mean a pure conductive polymer (PVA) that is not bonded to the conductive polymer 10 and the sugar alcohol 20 as in the above-described Experimental Examples.

Referring to FIG. 6, since the pure conductive polymer 10 (Experimental Example 1 (ex1)) and the pure water-soluble polymer (PVA) were not bonded to the sugar alcohol 20, it can be seen that a peak is low at a wavenumber of 500 cm⁻¹ or greater to 4,000 cm⁻¹ or less. Meanwhile, in Experimental Example 2 (ex2), as the conductive polymer 10 and the water-soluble polymer 30 are bonded, it can be seen that the peak is higher than those of Experimental Example 1 (ex1) and the pure water-soluble polymer (PVA) at the wavenumber in the above-described range. Meanwhile, in Experimental Example 7 (ex7), as the conductive polymer 10, the water-soluble polymer 30, and the sugar alcohol 20 are bonded, it can be seen that the peak is highest at the wavenumber in the above-described range.

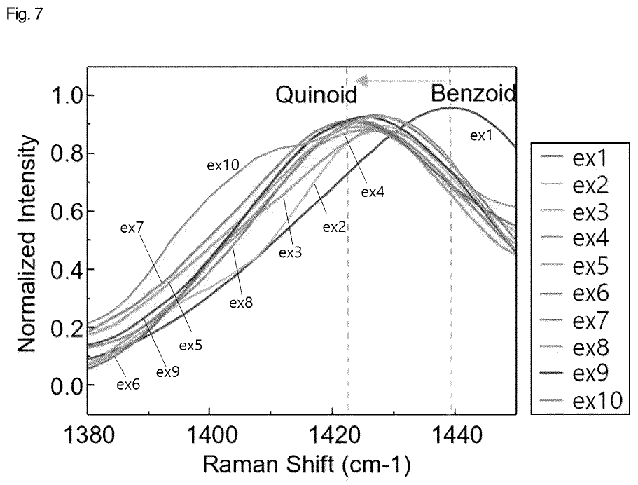

FIG. 7 is a Raman spectroscopy measurement graph of Experimental Examples 1 to 10 of the present application.

Referring to FIG. 7, in Experimental Examples 2 to 10 (ex2 to ex10), it can be seen that a Raman spectrum peak shifts to a wavenumber that is lower than a wavenumber of Experimental Example 1 (ex1) in a range of 1439 cm⁻¹ or greater to 1423 cm⁻¹ or less, which is defined as a C_{α}=C_{β} antisymmetric vibration peak.

Meanwhile, in the Raman spectrum shown in FIG. 7, the range including the peak of Experimental Example 1 (ex1) may mean a benzoid structure. That is, referring to FIG. 7, since the pure conductive polymer 10 is included in Experimental Example 1 (ex1), Experimental Example 1 (ex1) may have the benzoid structure.

Meanwhile, in the Raman spectrum shown in FIG. 7, the range including the peak of Experimental Examples 2 to 10 (ex2 to ex10) may mean a quinoid structure. That is, referring to FIG. 7, in Experimental Examples 2 to 10 (ex2 to ex10), since the conductive polymer 10 is bonded to the water-soluble polymer 30 and/or the sugar alcohol 20, the benzoid structure may be converted to have the quinoid structure meaning the expandable chain structure.

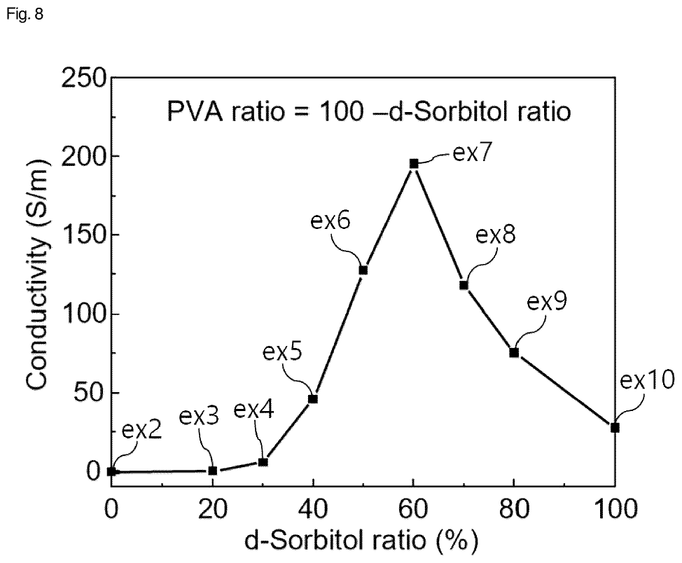

FIG. 8 is a conductivity measurement graph of Experimental Examples 2 to 10 of the present application.

Referring to FIG. 8, according to Experimental Examples 2 to 7 (ex2 to ex7), it can be seen that the conductivity is improved as the weight ratio of the sugar alcohol 20 increases, and the conductivity is the highest in Experimental Example 7 (ex7). Meanwhile, according to Experimental Examples 8 to 10 (ex8 to ex10), it can be seen that when the weight ratio of the sugar alcohol 20 increases more than that of Experimental Example 7 (ex7), the conductivity is rather deteriorated.

This may be because, according to Experimental Examples 2 to 7 (ex2 to ex7), as the weight ratio of the sugar alcohol 20 increases, it is converted into the expanded chain structure, so that a large amount of non-localized electrons exists along the linear conjugated pi electron system, and charge mobility is enhanced, thereby improving charge transport properties.

Meanwhile, the highest conductivity was shown in Experimental Example 7 (ex7) including the water-soluble polymer 30 and the sugar alcohol 20 in a weight ratio of 4:6, and accordingly, critical significance may be proved in the weight ratio of 4:6 of the water-soluble polymer 30 and the sugar alcohol 20.

Meanwhile, according to Experimental Examples 8 to 10 (ex8 to ex10), it can be seen that when the weight ratio of the sugar alcohol 20 increases more than that of Experimental Example 7 (ex7), the amount of the sugar alcohol 20 is excessively increased, and thus, non-localized electrons are reduced along the linear conjugated pi electron system, and charge mobility is deteriorated, thereby deteriorating charge transport properties.

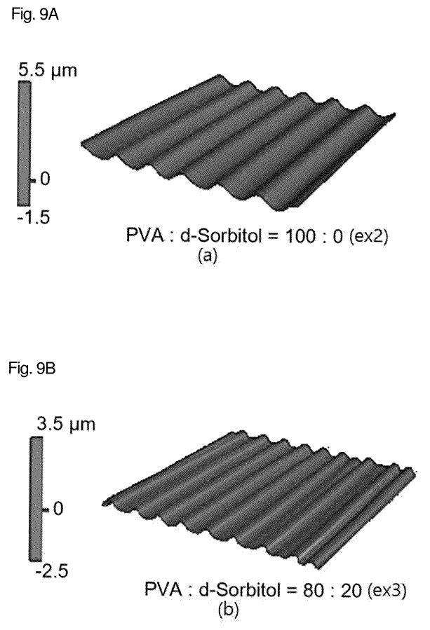

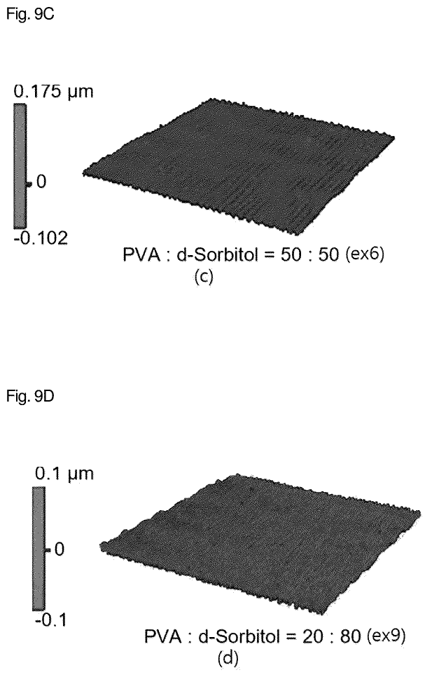

FIGS. 9A to 9D are surface profile data when Experimental Example 2, Experimental Example 3, Experimental Example 6, and Experimental Example 9 of the present application are stretched by 50%.

Referring to FIG. 9A, Experimental Example 2 (ex2) did not include the sugar alcohol 20, it was measured to have an average roughness of 906 nm, and exhibited surface profile characteristics of a brittle polymer having a large size or wrinkles and generating a lot of cracks.

Meanwhile, referring to Experimental Example 3 (ex3) shown in FIG. 9B, Experimental Example 6 (ex6) shown in FIG. 9C, and Experimental Example 9 (ex9) shown in FIG. 9D, the average roughness thereof was measured to be 120 nm, 65.8 nm, and 25.2 nm, respectively. Accordingly, it can be seen that as the weight ratio of the sugar alcohol 20 included in the biosignal measurement electrode 100 increases, the size of wrinkles is small and fine, and a small amount of cracks are generated, thereby having flexibility.

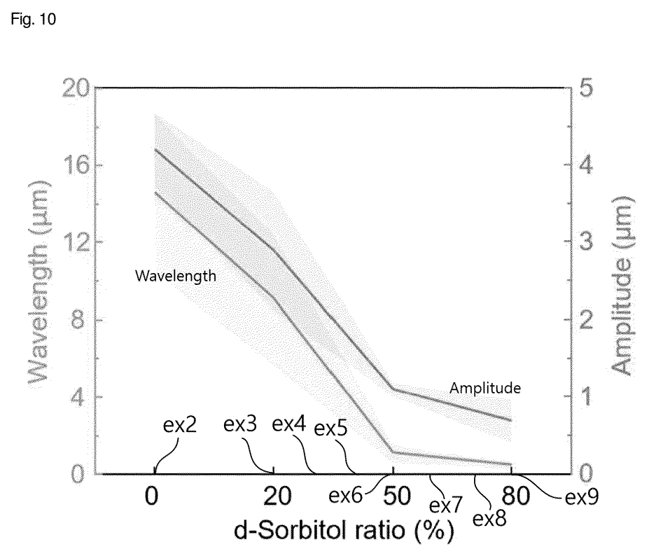

FIG. 10 is a graph of measuring a wavelength and an amplitude of wrinkles formed when Experimental Examples 2 to 9 of the present application are stretched by 50%.

Referring to FIG. 10, according to the experimental examples of the present application, it can be seen that as the weight ratio of the sugar alcohol 20 included in the biosignal measurement electrode 100 increases, the size of wrinkles is small and fine.

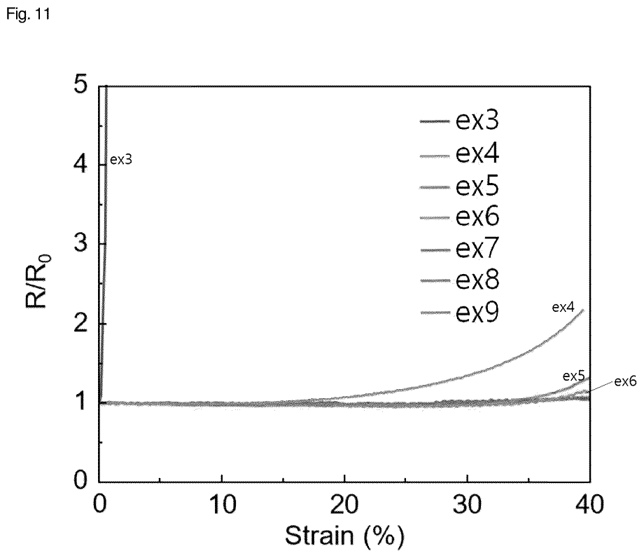

FIG. 11 is a graph of measuring a tensile strength of Experimental Examples 3 to 9 of the present application.

Referring to FIG. 11, in a case of Experimental Example 3 (ex3), since the weight of the sugar alcohol 20 is relatively the lowest compared to the weight of the water-soluble polymer 30, it can be seen that the flexibility is the lowest. In a case of Experimental Example 3 (ex3), cracks were generated on a surface according to a tension strength, and a rapid resistance change was shown, and Experimental Example 3 (ex3) did not increase by greater than 2%. In a case of Experimental Example 4 (ex4), cracks were not generated on a surface according to tension strength, and a resistance change was greater than double at a tension strength of 40%. In a case of Experimental Examples 5 and 6 (ex5 and ex6), the resistance change was about 20% at a tensile strength of 40%. In a case of Experimental Examples 7 and 8 (ex7 and ex8), the resistance change was hardly shown. In a case of Experimental Example 9 (ex9), since the weight of the water-soluble polymer 30 was relatively the lowest compared to the weight of the sugar alcohol 20, the adhesion was deteriorated, it is difficult to perform coating on the substrate sb, so that data could not be obtained.

Therefore, it can be seen that physical properties related to flexibility and adhesion of the biosignal measurement electrode 100 are changed according to the weight ratio of the water-soluble polymer 30 and the sugar alcohol 20 included in the biosignal measurement electrode 100.

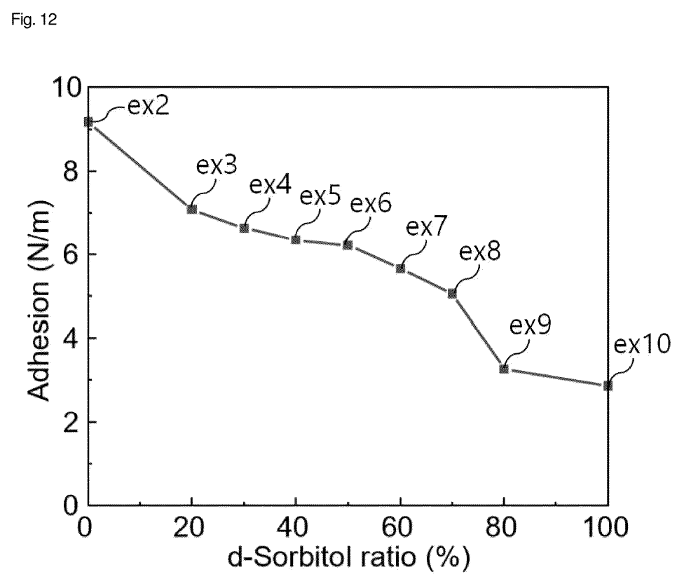

FIG. 12 is a graph showing test results of adhesion measured by attaching Experimental Examples 2 to 10 of the present application to the skin.

Referring to FIG. 12, according to Experimental Examples 2 to 10 (ex2 to ex10), it can be seen that the weight ratio of the sugar alcohol 20 relatively decreases, and the adhesion is enhanced as the weight ratio of the water-soluble polymer 30 relatively increases. This may be because, when the biosignal measurement electrode 100 is attached to the skin, the water-soluble polymer 30 is finely gelled by moisture (body fluid), and a hydrophilic functional group is stretched to enhance adhesion. That is, as the weight ratio of the water-soluble polymer 30 increases in the biosignal measurement electrode 100, the biosignal measurement electrode 100 may be substantially completely adhered to the fine curvature of the skin.

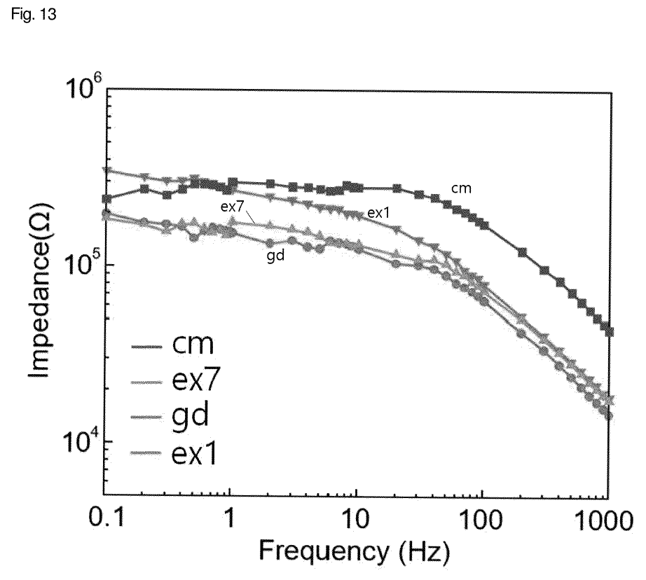

FIG. 13 is a graph showing impedance measured by attaching a commercially available electrode, Experimental Example 7 of the present application, a gold-based electrode, and Experimental Example 1 to the skin.

Referring to FIG. 13, it can be seen that an impedance value is relatively high in Experimental Example 1 (ex1), which does not include the commercially available electrode (cm), the water-soluble polymer 30, and the sugar alcohol 20. On the other hand, it can be seen that the impedance values in Experimental Example 7 (ex7) and the gold-based electrode (gd) are relatively low. Accordingly, it can be proved that Experimental Example 7 (ex7) has better signal quality than the commercially available electrode (cm).

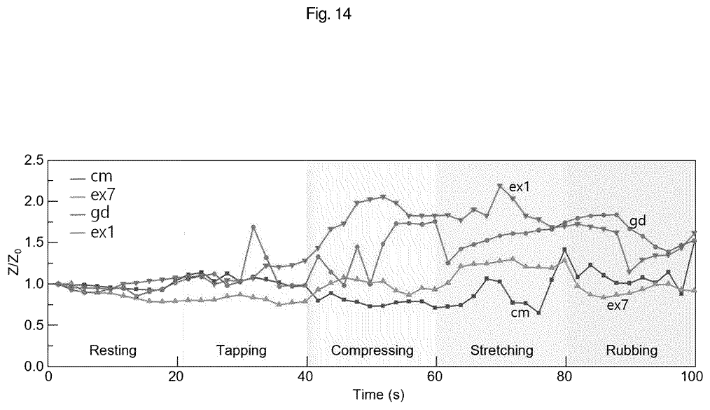

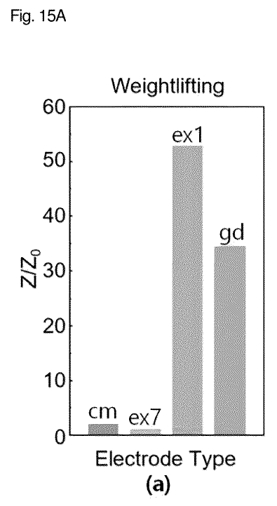

FIG. 14 is a graph showing impedance measured by attaching the commercially available electrode, Experimental Example 7 of the present application, the gold-based electrode, and Experimental Example 1 to the skin and applying various stimuli.

Referring to FIG. 14, various stimuli were applied to the commercially available electrode (cm), Experimental Example 7 (ex7), the gold-based electrode (gd), and Experimental Example 1 (ex1) through resting, tapping, pressing, stretching, and rubbing. In Experimental Example 1 (ex1), which does not include the commercially available electrode (cm), the water-soluble polymer 30, and the sugar alcohol 20, it can be seen that the impedance is relatively and greatly changed according to the various stimuli, and thus unstable. On the other hand, in Experimental Example 7 (ex7) and the gold-based electrode (gd), it can be seen that the impedance changes relatively small according to the various stimuli and is stable. Accordingly, it can be proved that Experimental Example 7 (ex7) has stable and excellent signal quality as compared to the commercially available electrode (cm).

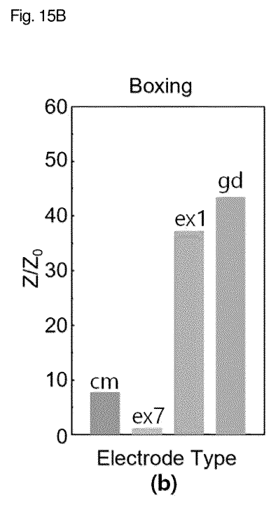

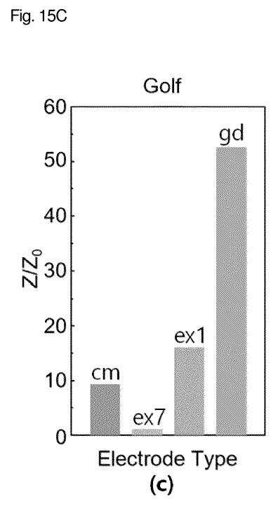

FIGS. 15A to 15C are graph showing changes in impedance measured by attaching the commercially available electrode, Experimental Example 7 of the present application, the gold-based electrode, and Experimental Example 1 to the skin and exercising.

Referring to FIGS. 15A to 15C, the commercially available electrode (cm), Experimental Example 7 (ex7), the gold-based electrode (gd), and Experimental Example 1 (ex1) were attached to a bicuspid muscle skin of the arm with a lot of muscle movement, and weightlifting (see FIG. 15A), boxing (see FIG. 15B), and golf (see FIG. 15C) were performed. Referring to FIGS. 15A to 15C, it can be seen that the impedance change is the lowest in Experimental Example 7 (ex7), which is lower than the commercially available electrode (cm). Meanwhile, in a case of the commercially available electrode (cm), sweat was generated in the skin by exercise, and the adhesion with the skin was weakened, and the impedance increased while being detached from the attached skin portion. In Experimental Example 1 (ex1) and the gold-based electrode (gd), stress was not tolerated, a part thereof was destroyed, and impedance was greatly increased after intense exercise. Accordingly, it can be proved that Experimental Example 7 (ex7) has stable and excellent signal quality as compared to the commercially available electrode (cm).

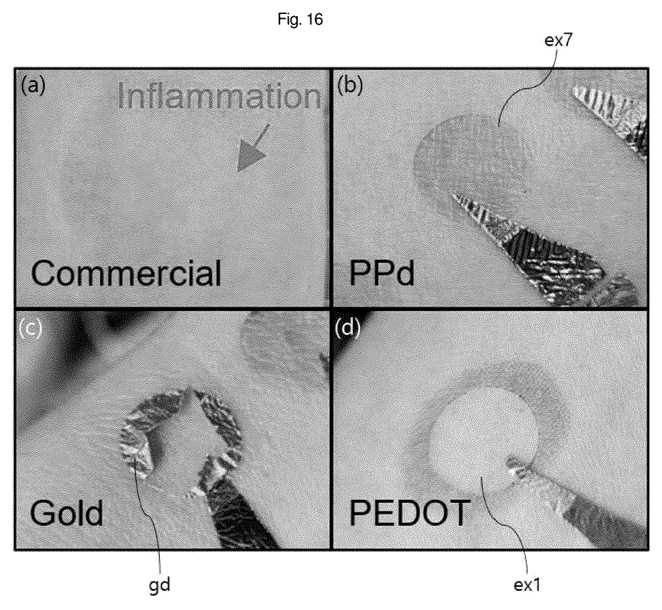

FIG. 16 is a photograph taken after attaching the commercially available electrode, Experimental Example 7 of the present application, the gold-based electrode, and Experimental Example 1 to the skin and exercising.

Referring to FIG. 16 (a), it can be seen that the commercially available electrode (cm) is detached from the skin after exercise. This may be because the adhesion of the used electrode (cm) is weak. Referring to FIG. 16(b), it can be seen that Experimental Example (ex7) is maintained without being detached from the skin or destroyed even after exercise. This may be because the adhesion and the flexibility of Experimental Example 7 (ex7) are excellent. Referring to FIGS. 16(c) and 16(d), it can be seen that the gold-based electrode (gd) and Experimental Example 1 (ex1) fail to withstand stress and are destroyed after exercise. This may be because the flexibility of the gold-based electrode (gd) and Experimental Example 1 (ex1) is low. Therefore, it can be proved that Experimental Example 7 (ex7) has excellent adhesion and flexibility.

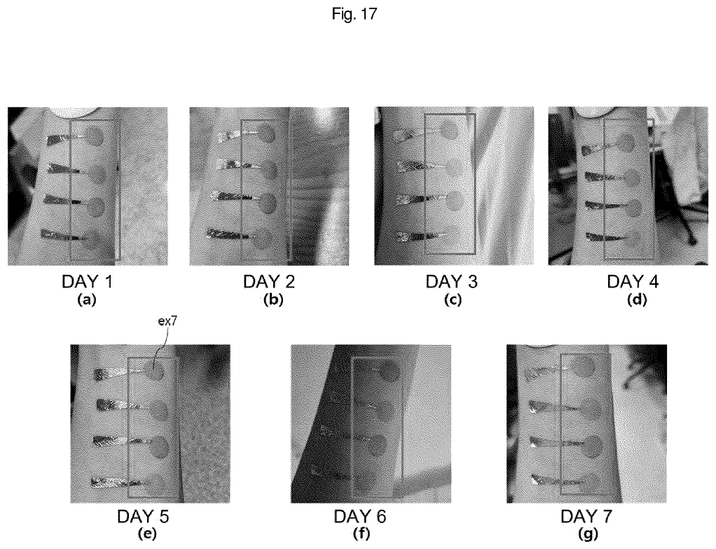

FIG. 17 is a photograph taken for 7 days while attaching Experimental Example 7 of the present application to the skin and living a daily life.

Referring to FIGS. 17(a) to 17(g), Experimental Example 7 (ex7) was attached to the skin and photographed for 7 days at intervals of 1 day. It can be seen that, even though the daily life was carried out for 7 days in a state where Experimental Example 7 (ex7) was attached to the skin, Experimental Example 7 (ex7) maintained a stable adhesion state with the skin. Therefore, it can be proved that Experimental Example 7 (ex7) has excellent adhesion.

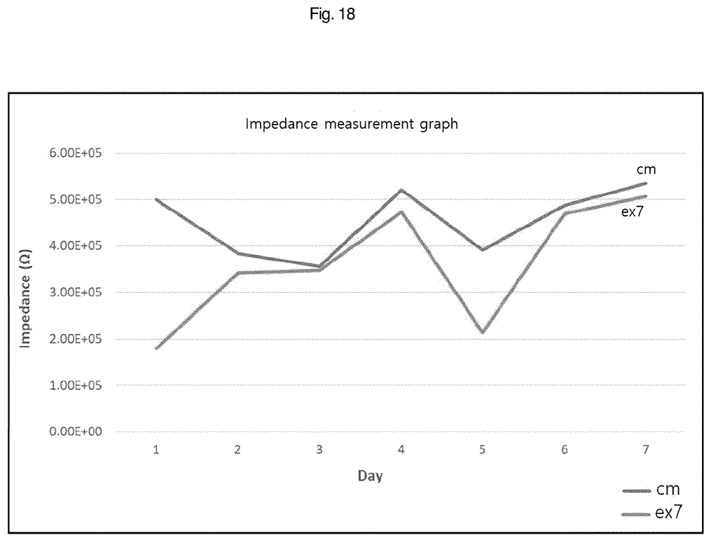

FIG. 18 is a graph of changes in impedance measured for 7 days by attaching Experimental Example 7 of the present application and the commercially available electrode to the skin and living a daily life.

Referring to FIG. 18, Experimental Example 7 (ex7) and the commercially available electrode (cm) were attached to the user's skin for 7 days, and the user performed daily life such as shower, exercise, and sleep for 7 days. As shown in FIG. 18, it can be seen that Experimental Example 7 (ex7) has a lower impedance value in daily life than the commercially available electrode (cm).

This may be because the adhesion and the flexibility of Experimental Example 7 (ex7) are excellent. Meanwhile, the commercially available electrode (cm) was attached to the skin for 7 days to cause rash and inflammation on the skin. However, Experimental Example 7 (ex7) was attached to the skin for 7 days to non-induce rash and inflammation on the skin, and thus excellent biocompatibility of Experimental Example 7 (ex7) can be proved.

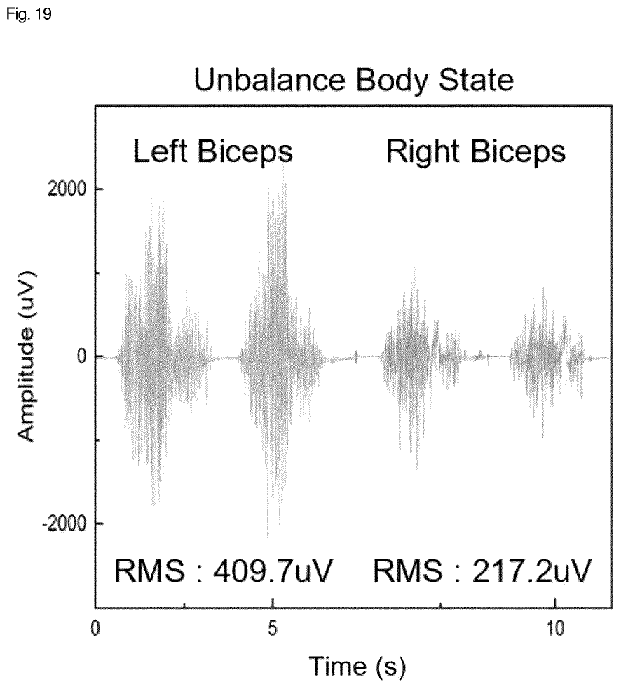

FIG. 19 is a graph of a biosignal measured by attaching Experimental Example 7 of the present application to the skin.

Referring to FIG. 19, Experimental Example 7 (ex7) was attached to the skin of the biceps of both arms, that is, the left biceps of the left arm and the right biceps of the right arm, and electrical signals were measured while holding dumbbells having different weights in both arms, respectively. As shown in FIG. 19, when dumbbells having different weights were lifted, different electrical signals could be obtained. Thus, biosignal measurement sensitivity of Experimental Example 7 (ex7) can be proved. In addition, since the act of lifting dumbbells requires intense muscle movement, an intense stimulus may also be applied to Experimental Example 7 (ex7) attached to the skin. Nevertheless, in Experimental Example 7 (ex7), the biosignal could be measured with excellent sensitivity due to excellent adhesion and flexibility. Thus, excellent adhesion and flexibility of Experimental Example 7 (ex7) can be proved.

While the present invention has been described in connection with the embodiments, it is not to be limited thereto but will be defined by the appended claims. In addition, it is to be understood that those skilled in the art can substitute, change, or modify the embodiments in various forms without departing from the scope and spirit of the present invention.

What is claimed is:

1. A method for producing a biosignal measurement electrode, the method comprising:
preparing a conductive polymer including a coil-type chain structure having a coil shape;
producing an electrode source including an expanded chain structure in which the coil-type chain structure is expanded and linearly arranged by providing a sugar alcohol and a water-soluble polymer to the conductive polymer;
forming a coating layer by providing the electrode source on a substrate; and
producing the biosignal measurement electrode by annealing the coating layer.

2. The method of claim 1,
wherein the sugar alcohol has a hydroxyl group, and a hydrogen bond with a non-conductive region of the conductive polymer is formed through the hydroxyl group to expand the coil-type chain structure.

3. The method of claim 2, wherein the conductive polymer of the expanded coil-type chain structure has an amorphous structure, and
the water-soluble polymer has a linear structure and is bonded with the conductive polymer having the expanded coil-type chain structure so as to linearly rearrange the coil-type chain structure according to the linear structure.

4. The method of claim 1, wherein when the biosignal measurement electrode is attached to a skin,
the water-soluble polymer is gelled and has a swelling force by reacting with a body fluid, and
the sugar alcohol forms a hydrogen bond with the conductive polymer and the water-soluble polymer to increase the swelling force of the water-soluble polymer and decrease a degree of gelation.

5. The method of claim 1, wherein in the preparing of the conductive polymer, the conductive polymer has a benzoid structure, and
in the producing of the electrode source, the benzoid structure is converted into a quinoid structure.

6. The method of claim 1, wherein the producing of the electrode source includes:
producing a mixed solution including 10 wt % or greater to 20 wt % or less of a mixture of the sugar alcohol and the water-soluble polymer; and
providing 10 wt % or greater to 30 wt % or less of the mixed solution to a solution in which the conductive polymer is dispersed in an amount of 0.1 wt % or greater to 3 wt % or less, and stirring the mixed solution.

7. The method of claim 6, wherein the sugar alcohol is provided in an amount of 50 wt % or greater to 70 wt % or less based on 100 wt % of the mixture.

8. A biosignal measurement electrode comprising:
a conductive polymer;
a sugar alcohol; and
a water-soluble polymer,
wherein the biosignal measurement electrode includes an expanded chain structure in which the conductive polymer, the sugar alcohol, and the water-soluble polymer are bonded, and
the expanded chain structure is more expanded than a coil-type chain structure having a coil shape of the conductive polymer before the bonding, and is linearly arranged.

9. The biosignal measurement electrode of claim 8,
wherein the expanded chain structure has a larger amount of non-localized electrons along a linear conjugated pi electromagnetic system in comparison with the coil-type chain structure.

10. The biosignal measurement electrode of claim 8, wherein when the biosignal measurement electrode is attached to a skin,
the water-soluble polymer forms a hydrogen bond with a body fluid to provide adhesion, and
the sugar alcohol forms a hydrogen bond with the conductive polymer and the water-soluble polymer to provide flexibility.

11. The biosignal measurement electrode of claim 8, wherein, in a range of 1439 cm⁻¹ or greater to 1423 cm⁻¹ or less, which is defined as a C_{α}=C_{β} antisymmetric vibration peak, a Raman spectrum peak shifts to a wavenumber that is lower than a wavenumber of the conductive polymer.

12. The biosignal measurement electrode of claim 8, wherein the water-soluble polymer and the sugar alcohol are included in a weight ratio of 4:6.

13. The biosignal measurement electrode of claim 8, wherein the conductive polymer includes poly3,4-ethylenedioxythiophene:polystyrene sulfonate,
the water-soluble polymer includes at least one of polyvinyl alcohol, polyethylene oxide, polyacryl amide, polyvinyl pyrrolidone, polyacrylic acid, polystyrenesulfonic acid, polysilicic acid, polyphosphoric acid, polyethylenesulfonic acid, polymaleic acid, polyamines, polyacrylamide, poly polyvinylpyrrolidone, and polyethylene glycol, and
the sugar alcohol includes at least one of sorbitol, ethylene glycol, glycerol, erythritol, threitol, arabitol, xylitol, ribitol, mannitol, galactitol, fucitol, iditol, inositol, volemitol, isomalt, maltitol, lactitol, maltotritol, maltotetraitol, and polyglycitol.

14. A biosignal measurement electrode comprising:
a first layer attached to a skin;
a second layer stacked on the first layer; and
a third layer stacked on the second layer,
wherein the first to third layers include an expanded chain structure in which a conductive polymer, a sugar alcohol, and a water-soluble polymer are bonded, and
the expanded chain structure is more expanded than a coil-type chain structure having a coil structure of the conductive polymer before the bonding, and is linearly arranged.

15. The biosignal measurement electrode of claim 14, wherein the first layer includes a larger amount of the water-soluble polymer in comparison with the second layer and the third layer, and includes a smaller amount of the sugar alcohol in comparison with the second layer and the third layer,
the second layer includes a smaller amount of the water-soluble polymer in comparison with the first layer and a larger amount of the water-soluble polymer in comparison with the third layer, and includes a larger amount of the sugar alcohol in comparison with the first layer and a smaller amount of the sugar alcohol in comparison with the third layer, and
the third layer includes a smaller amount of the water-soluble polymer in comparison with the first layer and the second layer, and includes a larger amount of the sugar alcohol in comparison with the first layer and the second layer.
